(19) United States
(12) Patent Application Publication
(10) Pub. No.: US 2015/0157647 A1
(43) Pub. Date: Jun. 11, 2015

(54) METHODS AND COMPOSITIONS RELATED TO ACUTE RESPIRATORY DISTRESS SYNDROME

(71) Applicant: Jules B. Puschett, Houston, TX (US)

(72) Inventor: Jules B. Puschett, Houston, TX (US)

(21) Appl. No.: 14/566,427

(22) Filed: Dec. 10, 2014

Related U.S. Application Data
(60) Provisional application No. 61/914,007, filed on Dec. 10, 2013.

(51) Int. Cl. A61K 31/585; G01N 33/543

(52) U.S. Cl. CPC A61K 31/585; G01N 33/54306; G01N 2800/52

(57) ABSTRACT

Certain embodiments are directed to methods of identifying patients with ARDS by detecting elevated levels of marinobufagenin (MBG) and treating the same with anti-MBG agents.

This application claims priority to U.S. Provisional Application Ser. No. 61/914,007 filed Dec. 10, 2013, which is incorporated herein by reference in its entirety.

### BACKGROUND

Acute respiratory distress syndrome (ARDS) is a severe, and often life-threatening complication of several systemic disorders and direct injury to the lungs. It is associated with a high mortality rate, primarily as a consequence of multiple organ failure (Frutos-Vivar et al., Curr Opin Crit Care 2004, 10:1-6). ARDS occurs when fluid builds up in the alveoli of the lungs. More fluid in the lungs means less oxygen reaching the bloodstream, which deprives organs of the oxygen they need to function. ARDS typically occurs in people who are already critically ill or who have significant injuries. Symptoms of ARDS includes severe shortness of breath, labored and unusually rapid breathing, low blood pressure, and confusion and extreme tiredness, which usually develops within a few hours to a few days after an original disease or trauma. The risk of death increases with age and severity of illness. Of those that survive ARDS, some recover completely while others experience lasting damage to their lungs.

There is no specific test to identify ARDS. A diagnosis is reached by ruling out other diseases and conditions—for example, certain heart problems—that can produce similar symptoms. A chest X-ray can reveal which parts of the lungs have fluid and whether the heart is enlarged. CT scans can provide detailed information about the structures within the heart and lungs. Arterial blood can be used to measure oxygen levels. Other types of blood tests can check for signs of infection or anemia. Because the signs and symptoms of ARDS are similar to those of certain heart problems, a physician may recommend cardiac assessments such as: electrocardiogram or echocardiogram.

Despite 40 years of research and advances in medical technology, ARDS-associated mortality remains high, and no pharmacological therapies effectively improve its clinical course. Four decades after the first description of ARDS and followed by multiple studies on this syndrome, the only approach that has been proven to decrease mortality is mechanical ventilation with a low tidal volume (The Acute Respiratory Distress Syndrome Network. N Engl J Med 2000, 342:1301-08).

### SUMMARY

Bufadienolides were discovered in amphibians. They are thought to act by virtue of their ability to inhibit Na+/K+-ATPase activity (Flier et al., Science 1980, 208:503-05). Several bufadienolides have been suggested as candidate sodium pump ligands (SPLs) in mammals, including marinobufagenin (MBG), which acts in vitro as a vasoconstrictor (Fedorova et al., Am. J. Hypertens. 1997, 10:929-35; Lopatin et al., J. Hypertens. 1999, 17:1179-87). Enhanced MBG production occurs in pathological states associated with fluid retention, including essential and salt-sensitive hypertension, preeclampsia, and uremic cardiomyopathy (Gonick et al, Clin. Exp. Hypertens. 1998, 20: 617-27, Bagrov et al., Hypertension 1998, 31:1097-1103; Fedorova et al., Hypertension 2001, 37:462-66; Lopatin et al., J. Hypertens. 1999, 17:1179-87; Fedorova et al, Circulation 2002; 105: 1122-27; Kennedy et al., Hypertension 2006, 47:448-495).

The inventor has determined that marinobufagenin (MBG) is involved in the production of the vascular leak seen in acute respiratory distress syndrome (ARDS). The hyper-permeability of the endothelium of the lung vasculature can be associated with impaired function of the “tight junctions” of the lung endothelium. The inventor has discovered that MBG levels are elevated in the urine and blood of patients having ARDS. In urine MBG averages 306 pg/mg creatinine in non-ARDS ICU patients. MBG in ARDS patient's urine was elevated to 753 pg MBG/mg creatinine Blood levels in non-ARDS ICU patients was 37.5 pg/ml vs. 63.2 pg/ml in ARDS patients. Thus, MBG is a diagnostic agent for ARDS and can be used to identify those patients having or developing ARDS. An MBG determination will allow medical personnel to administer prophylactic treatment as early as possible and distinguish ARDS from conditions presenting similar symptoms.

Most people who develop ARDS are already hospitalized for another condition, and many are critically ill. A subject is at risk of developing ARDS if the subject has an infection or sepsis. Also, people who have a history of chronic alcoholism are at higher risk of developing ARDS. The mechanical cause of ARDS is fluid leaked from the smallest blood vessels in the alveoli. Normally, a protective membrane keeps this fluid in the vessels. Severe illness or injury, however, can cause inflammation that undermines the membrane's integrity, leading to the fluid leakage. Severe illnesses that can cause ARDS include, but are not limited to: sepsis, hemorrhagic shock, acute myocardial infarction, or acute kidney injury.

In certain aspects three vascular beds can experience permeability changes when exposed to MBG: the lung, blood brain barrier, and the splanchnic vascular bed (i.e., the gastric, small intestinal, colonic, pancreatic, hepatic, and splenic circulations). The splanchnic vascular bed includes the bowel, which is associated with the omentum (a large fold of visceral peritoneum that extends from the greater curvature of the stomach). Vascular permeability can be modeled in vitro using endothelial monolayer assays. Studies performed using endothelial monolayers obtained from rat lung demonstrate that MBG causes vascular “leak” by interfering with the function of the adherence proteins (Uddin et al., Am J Physiol Regul Integr Comp Physiol 296: R1726-R1734, 2009).

Certain embodiments are directed to measuring MBG in patients having ARDS, suspected of having ARDS (i.e., patients demonstrating symptoms that indicate ARDS), or at risk of developing ARDS. In certain aspects a patient is identified as being at risk of developing ARDS by detecting an elevated level of MBG in the urine or blood. Once a patient is identified as “at risk for ARDS,” as having ARDS, or is in the early stages of developing ARDS, prophylactic or therapeutic treatments are administered to the patient. In certain aspects the prophylactic treatment is administration of an MBG antagonist. One such MBG antagonist is resibufagenin (RBG). Other treatments for ARDS include therapies that increase oxygen levels of the subject and/or providing supportive care. A primary goal of ARDS therapy is to provide oxygen to the lungs and other organs (such as the brain and kidneys). Oxygen usually is given through nasal prongs or a mask that fits over the mouth and nose. However, if oxygen levels do not increase oxygen can be administered through a breathing tube connected to a ventilator. Supportive care can include, but is not limited to medications to relax, relieve discomfort, and treat pain; ongoing monitoring of heart and lung function; nutritional support; anti-microbial therapies to treat infection; blood clot prevention therapies (blood thinners and/or compression dressings); medications to inhibit intestinal bleeding; and the like.

Certain aspects are directed to methods of identifying a patient suffering from, developing early stage ARDS, or at risk of developing ARDS comprising determining or measuring the level of MBG in a sample from the patient. In certain aspects the sample is blood or urine. If the levels of MBG are elevated then the patient will be identified as having ARDS, at risk of developing ARDS, or is in early stages of developing ARDS. In certain aspects elevated levels of MBG are when the level is at least 1.5 times an MBG reference level. In a further aspect elevated levels of MBG are when the urine level is at least 1.5 times an MBG reference level. In certain aspects the blood or urine levels are at least or about 1.5, 2.0, or 3.0 times the levels of a patient not having ARDS or a reference level for MBG. In other aspects, elevated levels of MBG levels are at least 30, 40, 50, 60, 70, 80, 90, 100, 200% or more than normal levels or a reference level. In other aspects elevated MBG levels are at least 1.5, 2, 3, or more time the normal levels or a reference level. In certain aspects the MBG levels are measured using an ELISA assay, lateral flow, colloidal gold, nanotechnology (e.g., microfluidics), or other analytical techniques.

MBG is elevated in other syndromes such as preeclampsia and traumatic brain injury. It has been shown that the administration of the antagonist of MBG, resibufagenin (RBG) is therapeutically effective. RBG administration results in an amelioration of tissue injury in a rodent brain contusion model when given 1 hour after the insult and prevents preeclampsia in animal models. RBG antagonizes MBG activity and may be an effective treatment for ARDS, traumatic brain injury, and preeclampsia. Certain embodiments are directed to treating an ARDS patient by administering an MBG antagonist or anti-MBG agent. In certain aspects the anti-MBG agent is resibufagenin (RBG). The anti-MBG agent can be provided in conjunction with another standard ARDS therapy.

Other embodiments of the invention are discussed throughout this application. Any embodiment discussed with respect to one aspect of the invention applies to other aspects of the invention as well and vice versa. Each embodiment described herein is understood to be embodiments of the invention that are applicable to all aspects of the invention. It is contemplated that any embodiment discussed herein can be implemented with respect to any method or composition of the invention, and vice versa. Furthermore, compositions and kits of the invention can be used to achieve methods of the invention.

The use of the word “a” or “an” when used in conjunction with the term “comprising” in the claims and/or the specification may mean “one,” but it is also consistent with the meaning of “one or more,” “at least one,” and “one or more than one.”

Throughout this application, the term “about” is used to indicate that a value includes the standard deviation of error for the device or method being employed to determine the value.

The use of the term “or” in the claims is used to mean “and/or” unless explicitly indicated to refer to alternatives only or the alternatives are mutually exclusive, although the disclosure supports a definition that refers to only alternatives and “and/or.”

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### DESCRIPTION

Acute respiratory distress syndrome (ARDS) is a serious, often fatal condition in patients leading to their admission to the Intensive Care Unit (ICU) of the hospital. It is characterized by leakage of fluid through a damaged inner lining (the endothelium) of blood vessels in the lung. This results in diminished oxygenation of the blood, which if not corrected expeditiously results in multiple organ failure and, consequently, death of the patient. This condition is often seen in patients who have suffered sepsis, often leading to shock. Despite strenuous efforts to treat and alleviate matters, the mortality rate in such patients remains at 41-58 percent.

### I. Methods of Detecting Marinobufagenin (MBG)

Marinobufagenin is a steroid having a structure illustrated in Formula I.

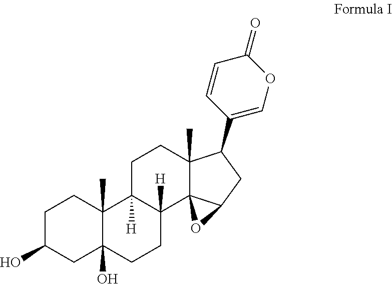

MBG can be detected using a variety of assays including, but not limited to immuno-detection, microchip, or lateral flow based methods. Immuno-based assays include, but are not limited to radiolabeled, enzyme, fluorescence, dot blot, chemiluminescence, dip-stick, or biosensor assays. See, for example, Principles and Practice of Immunoassays, Price, C. P. and Newman, D. J. (Eds.), Stockton Press, 1997; The Immunoassay Handbook; 2nd Edition, Wild, D. (Ed.), Nature Publishing Group, London, 2001.

Methods for measuring MBG in the blood or urine can provide sensitivities in the pg/ml (picograms per milliliter) range or pg/mg creatinine, respectively. In certain aspects the MBG detection methods employ an MBG specific ELISA assay. In certain aspects the MBG assay discriminates between MBG and resibufogenin (RBG). In certain aspects, antibodies are used to detect the presence of MBG in an original or processed sample obtained from a subject (Abi-Ghanem et al., Journal of Immunoassay and Immunochemistry, 32:31-46, 2011). Samples obtained from a subject may include, for example, cells, tissue, blood, serum, or urine. For example, a sample can be blood or urine collected from a subject. A sample can be analyzed directly or extracted before analysis.

In certain aspects a sample is contacted with an effective amount of one or more antibody/antibodies and the sample is screened to detect an antibody-MBG complex, such as detecting the binding reaction between the antibody and the MBG. Detection of an antibody-antigen complex or binding reaction indicates that the sample contains MBG. In some embodiments, the one or more antibody is labeled with a detectable moiety, such as a fluorescent label, so that its signal changes upon binding to MBG.

In other embodiments, MBG in the sample is immobilized on a surface and detected. In certain aspects MBG is immobilized prior to introduction of the labeled antibody, and the amount of the signal, corresponding to the amount of bound labeled antibody, correlates to the amount of MBG in the sample. In still other embodiments, MBG is captured by an immobilized unlabeled first antibody, after which a labeled second antibody is introduced to bind to the captured MBG and produce a signal in proportion to the amount of captured MBG. In other embodiments the binding of MBG is detected by altering the properties of the surface to which it is directly or indirectly bound.

In general, an antibody can be used as a labeled primary reagent in a direct assay or as an unlabeled reagent to be detected by a secondary developing antibody conjugate, such as labeled anti-rabbit antibody, in an indirect assay (Abi-Ghanem et al., Journal of Immunoassay and Immunochemistry, 32:31-46, 2011). Additionally, an antibody can be used in a competition assay to detect an antigen or antibody in a sample. For example, MBG in a sample extract is captured by an unlabeled antibody immobilized on the surface of an ELISA well and then detected by a labeled antibody of the same or different kind and/or specificity. Alternatively, the sample can be suspended in a buffer and mixed directly with an antibody, thus allowing the antibody to form an immune complex with MBG. The reduction of free antibody due to complex formation can then be determined in a second step, based on solid-phase ELISA with purified MBG, by comparing the relative reactivity of free residual antibody left over after sample incubation (sample reactivity) to that of the same antibody when not mixed with the sample (reference reactivity). The ratio of sample to reference antibody reactivity will be inversely proportional to the amount of MBG in the sample.

In certain aspects, methods of the invention can be adapted for lateral flow assays and devices supporting such. Lateral flow assays, also known as immunochromatographic assays, are typically carried out using a simple device intended to detect the presence (or absence) of a target analyte in the sample. Most commonly these tests are used for medical diagnostics either for home testing, point of care testing, or laboratory use. Often produced in a dipstick format, these assays are a form of immunoassay in which the test sample flows along a solid substrate via capillary action. After the sample is applied to the test it encounters a colored or labeling reagent which mixes with the sample and transits the substrate encountering lines or zones which have been pretreated with an antibody or antigen or affinity reagent. Depending upon the analyte present in the sample the colored or labeling reagent can become bound at the test line or zone. Lateral flow assays can operate as either competitive or sandwich assays.

In still a further aspect, methods of the invention can be adapted for protein array assays and devices supporting such. Protein arrays or microarrays (also known as a biochip, or a proteinchip) are measurement devices used in biomedical applications to determine the presence and/or amount of an analyte(s) in biological samples, e.g. blood, urine, swabs, tissues scrappings, etc. Typically, a number of different capture agents, most frequently monoclonal antibodies, can be deposited on a chip surface (glass or silicon) in an array. This format is also referred to as a microarray (a more general term for chip based biological measurement devices).

In yet another aspect the assay can be a microfluidic chip-based assay. Microfluidics is an important innovation in biochip technology. Since microfluidic chips can be combined with immunodetection and mass spectrometric analysis (See Wang et al., Lab Chip 13:4190-97, 2013; Baker et al. Bioanalysis 1(5): 967-75 2009; Wang et al., Anal. Chem. 72:832-839, 2000).

### II. Anti-MBG Compositions

In certain embodiments, the invention also provides compositions comprising an anti-MBG agent or MBG antagonist with one or more of the following: a pharmaceutically acceptable diluent, a carrier, a solubilizer, an emulsifier, and/or a preservative. Such compositions may contain an effective amount of at least one anti-MBG agent. The use of an anti-MBG agent in combination with other ARDS or respiratory therapy is also included. Standard therapies primarily strive to improve the levels of oxygen in the blood. To increase blood oxygenation a subject can be administered supplemental oxygen through a mask that fits tightly over the nose and mouth or by mechanical ventilation. A mechanical ventilator pushes air into the lungs and forces some of the fluid out of the air sacs. In certain aspects ARDS treatment includes carefully managing the amount of intravenous fluids. Too much fluid can increase fluid buildup in the lungs and too little fluid can put a strain on the heart and other organs, and lead to shock. ARDS patients may be medicated to prevent and treat infections, relieve pain and discomfort, prevent clots in the legs and lungs, minimize gastric reflux, and treat other symptoms or sequelae.

In one aspect an anti-MBG agent is resibufagenin (RBG; epoxy-3β-hydroxy-5β-bufa-20,22-dienolide). Resibufagenin has the structure shown in Formula II. Other MBG antagonists can be produced that maintain the structural context of the hydrogen at the 5 position of resibufagenin.

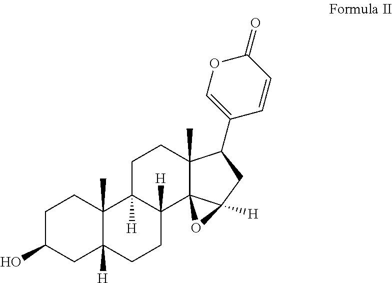

The anti-MBG agents may be formulated into therapeutic compositions in a variety of dosage forms such as, but not limited to, liquid solutions or suspensions, tablets, pills, powders, suppositories, polymeric microcapsules or microvesicles, liposomes, and injectable or infusible solutions. The preferred form depends upon the mode of administration and the particular disease targeted. The compositions also preferably include pharmaceutically acceptable vehicles, carriers, or adjuvants, well known in the art.

Acceptable formulation components for pharmaceutical preparations are nontoxic to recipients at the dosages and concentrations employed. In addition to the anti-MBG agents that are provided, compositions may contain components for modifying, maintaining, or preserving, for example, the pH, osmolarity, viscosity, clarity, color, isotonicity, odor, sterility, stability, rate of dissolution or release, adsorption, or penetration of the composition. Suitable materials for formulating pharmaceutical compositions include, but are not limited to, amino acids (such as glycine, glutamine, asparagine, arginine or lysine); antimicrobials; antioxidants (such as ascorbic acid, sodium sulfite or sodium hydrogen-sulfite); buffers (such as acetate, borate, bicarbonate, Tris-HCl, citrates, phosphates or other organic acids); bulking agents (such as mannitol or glycine); chelating agents (such as ethylenediamine tetraacetic acid (EDTA)); complexing agents (such as caffeine, polyvinylpyrrolidone, beta-cyclodextrin or hydroxypropyl-beta-cyclodextrin); fillers; monosaccharides; disaccharides; and other carbohydrates (such as glucose, mannose or dextrins); proteins (such as serum albumin, gelatin or immunoglobulins); coloring, flavoring and diluting agents; emulsifying agents; hydrophobic polymers (such as polyvinylpyrrolidone); low molecular weight polypeptides; salt-forming counter ions (such as sodium); preservatives (such as benzalkonium chloride, benzoic acid, salicylic acid, thimerosal, phenethyl alcohol, methylparaben, propylparaben, chlorhexidine, sorbic acid or hydrogen peroxide); solvents (such as glycerin, propylene glycol or polyethylene glycol); sugar alcohols (such as mannitol or sorbitol); suspending agents; surfactants or wetting agents (such as pluronics, PEG, sorbitan esters, polysorbates such as polysorbate 20, polysorbate 80, triton, tromethamine, lecithin, cholesterol, tyloxapal); stability enhancing agents (such as sucrose or sorbitol); tonicity enhancing agents (such as alkali metal halides, preferably sodium or potassium chloride, mannitol sorbitol); delivery vehicles; diluents; excipients and/or pharmaceutical adjuvants. (see Remington's Pharmaceutical Sciences, 18 th Ed., (A. R. Gennaro, ed.), 1990, Mack Publishing Company), hereby incorporated by reference.

Formulation components are present in concentrations that are acceptable to the site of administration. Buffers are advantageously used to maintain the composition at physiological pH or at a slightly lower pH, typically within a pH range of from about 4.0 to about 8.5, or alternatively, between about 5.0 to 8.0. Pharmaceutical compositions can comprise TRIS buffer of about pH 6.5-8.5, or acetate buffer of about pH 4.0-5.5, which may further include sorbitol or a suitable substitute therefor.

The pharmaceutical composition to be used for in vivo administration is typically sterile. Sterilization may be accomplished by filtration through sterile filtration membranes. If the composition is lyophilized, sterilization may be conducted either prior to or following lyophilization and reconstitution. The composition for parenteral administration may be stored in lyophilized form or in a solution. In certain embodiments, parenteral compositions are placed into a container having a sterile access port, for example, an intravenous solution bag or vial having a stopper pierceable by a hypodermic injection needle, or a sterile pre-filled syringe ready to use for injection.

The above compositions can be administered using conventional modes of delivery including, but not limited to, intravenous, intraperitoneal, oral, intraarterial, intrapleural, intrathecal, and by infusion through a regional catheter. In certain aspects an anti-MBG agent will be administered intravenously, or by inhalation or instillation into the lungs. When administering the compositions by injection, the administration may be by continuous infusion or by single or multiple boluses. For parenteral administration, the anti-MBG agents may be administered in a pyrogen-free, parenterally acceptable aqueous solution comprising the desired anti-MBG agents in a pharmaceutically acceptable vehicle. A particularly suitable vehicle for parenteral injection is one in which one or more anti-MBG agents are formulated as a sterile, isotonic solution, properly preserved.

Once the pharmaceutical composition of the invention has been formulated, it may be stored in sterile vials as a solution, suspension, gel, emulsion, solid, or as a dehydrated or lyophilized powder. Such formulations may be stored either in a ready-to-use form or in a form (e.g., lyophilized) that is reconstituted prior to administration.

If desired, stabilizers that are conventionally employed in pharmaceutical compositions, such as sucrose, trehalose, or glycine, may be used. Typically, such stabilizers will be added in minor amounts ranging from, for example, about 0.1% to about 0.5% (w/v). Surfactant stabilizers, such as TWEEN®-20 or TWEEN®-80 (ICI Americas, Inc., Bridgewater, N.J., USA), may also be added in conventional amounts.

The components used to formulate the pharmaceutical compositions are preferably of high purity and are substantially free of potentially harmful contaminants (e.g., at least National Food (NF) grade, generally at least analytical grade, and more typically at least pharmaceutical grade). Compositions for parental administration are also sterile, substantially isotonic and made under GMP conditions.

For the compounds of the present invention, alone or as part of a pharmaceutical composition, such doses are between about 0.001 mg/kg and 1 mg/kg body weight, preferably between about 1 and 100 μg/kg body weight, most preferably between 1 and 10 μg/kg body weight.

Therapeutically effective doses will be easily determined by one of skill in the art and will depend on the severity and course of the disease, the patient's health and response to treatment, the patient's age, weight, height, sex, previous medical history and the judgment of the treating physician.

### III. Methods of Matching Hypertension Therapy Using MBG Levels

Essential hypertension is a term that refers to patients with high blood pressure who do not have secondary causes (e.g., kidney failure or insufficiency, pheochromocytoma, primary hyperaldosteronism, etc.) and accounts for 90-95% of all patients presenting for evaluation of elevated blood pressure. Patients with essential hypertension can be subcategorized as to whether the disorder is primarily related to volume expansion (too much salt and water in the body) or to vasoconstriction. The ability to determine the etiology of hypertension in an outpatient (or an inpatient) setting when a patient is first identified as hypertensive can provide a more rapid and accurate assignment of that patient to a therapy most likely to be effective in that particular patient. This would replace current therapeutic guesswork and would result in a diagnostic/therapeutic “match,” an example of “personalized medicine.” Accordingly, patients could be correctly categorized as having volume expansion-mediated or vasoconstrictive hypertension or both.

Hypertension currently affects about 30% of the US population and many times that number worldwide. While the current approximation of the proportions of patients who have the volume expansion variety of hypertension is approximately 30-40% of the hypertensive population, while the group suffering from vasoconstriction hypertension is about 60-70%. Those figures are rapidly changing because patients who have a propensity to develop the volume expansion-mediated form of elevated blood pressure include: (1) the elderly, (2) the obese, (3) African-Americans (4) Hispanics and (5) a subset of type II diabetics. Notably, our population is aging, there is an epidemic of obesity and diabetes in this country, and African-Americans and Hispanics are an increasing percentage of the population. Thus, it is postulated that in the next 20-30 years, the larger percentage of patients with the volume expansion type of hypertension will exceed those with the vasoconstrictive form, reversing the current proportions.

There are currently no diagnostic methods to determine which patient has the volume expansion form and which has the vasoconstrictive basis of his/her hypertension. The inventor has conceived a rapid, easily performed test of hypertension so that the correct therapeutic agent might be administered when the correct etiologic diagnosis is determined at the patient's first visit. This determination is made on the following bases:

Marinobufagenin (MBG) is a member of a class of substances called the bufodienolides. It is also classified as a “cardiotonic steroid” or cardiac glycoside (Kennedy et al. Hypertension 47(3):488-93, 2006). Evidence has accumulated which indicates that the level of MBG in the blood and/or urine is a reflection of body volume (Puschett et al., Biochimica et Biophysica Acta 1802:1246-53, 2010; Lichardus and Pearce, Nature 5021:407-409, 1966). In certain aspects an elevation in the amount of MBG is indicative of the patient suffering from an expansion of his body fluid volume. Thus, the patient having elevated levels of MBG will be administered an agent directed toward reducing body volume (e.g., a diuretic) or an agent that antagonizes MBG. In certain aspects an MBG antagonist is resibufogenin (RBG), another member of the bufodienolide group of compounds.

With regard to vasoconstriction, the major agent found to subserve this function in the body's vasculature is angiotensin, which is produced by the kidney (Reviewed in Kobori et al., Pharmacological Reviews 59:251-87, 2007). Angiotensin is metabolized and produces a metabolite called angiotensinogen. The levels of angiotensinogen are indicative of the activity of the renin-angiotensin system in the body. The activity of the renin-angotensin system in the body can be measured by determining the amount of angiotensinogen excreted in the urine. Accordingly, a simplified, reproducible test for the simultaneous determination of MBG and angiotensinogen can be utilized to determine which form of hypertension exists in the individual patient by detecting MBG and angiotensinogen in a sample from a patient, allowing the appropriate treatment to be prescribed based upon categorization of a patient. A test kit can be provided and made available in the physician's office, in the clinic, in the field, or in a hospital laboratory.

In certain aspects a patient is tested and is positive for elevated levels of both MBG and angiotensinogen, then the patient is administered an anti-hypertensive therapy that includes agents for treating volume expansion hypertension and vasoconstrictive hypertension. In the case where MBG is elevated and angiotensinogen is not elevated then a patient is administered a therapeutic agent(s) to treat a volume expansion form of hypertension. In the event that MBG levels are not elevated and angiotensinogen levels are elevated then a patient is administered a therapeutic agent(s) to treat vasoconstrictive hypertension. A non-limiting example of a therapy for vasoconstrictive hypertension includes administration of an angiotensin converting enzyme (ACE) inhibitor and/or calcium channel blocker. A non-limiting example of a volume expansion hypertension therapy includes administration of a diuretic and/or a MBG antagonist.

In certain aspects, elevated levels of MBG or angiotensinogen are at least 30, 40, 50, 60, 70, 80, 90, 100, 200% or more than normal levels or a reference level. In other aspects elevated MBG or angiotensinogen levels are at least 1.5, 2, 3, or more time the normal levels or a reference level. In certain embodiments MBG and angiotensinogen are detected or measured in blood or urine.

In an effort to better understand the mechanisms involved in human ARDS an animal model of ARDS in the rat was used. MBG was measured in the serum of sham rats and rats with an induced ARDS-like syndrome. In the “ARDS” animals there is a fourfold increase in serum MBG from that obtained in the sham animals. The table below summarizes the data from such a study.

Rat serum

| | MBG (pg/ml) |
|---|---|
| Control | |
| Rat 1 | 8.5 |
| Rat 2 | 10.1 |
| Rat 3 | 10.9 |
| Rat 4 | 10.0 |
| Rat 5 | 7.5 |
| Mean | 9.4 |
| ARDS | |
| Rat 6 | 40.2 |
| Rat 7 | 46.7 |
| Rat 8 | 26.7 |
| Rat 9 | 23.2 |
| Rat 10 | 36.4 |
| Mean | 34.6 |

1. A method of identifying a patient at risk of developing acute respiratory distress syndrome (ARDS) comprising determining marinobufagenin (MBG) level in blood or urine of a patient suspected of having ARDS or is at risk of developing ARDS; and identifying the patient as in need for ARDS therapy when a blood level of MBG is at least 50% greater than a reference level or a urine level is at least 50% greater than a reference level.

2. The method of claim 1, further comprising administering an ARDS treatment to a subject having elevated levels of MBG.

3. The method of claim 1, wherein the MBG level is measured using an ELISA assay.

4. The method of claim 1, wherein the MBG level is measured using a lateral flow assay.

5. The method of claim 1, wherein an elevated blood level of MBG is greater than 45 pg/ml or elevated urine level is greater than 450 pg MBG/mg creatinine

6. The method of claim 1, wherein an at risk subject is diagnosed with sepsis.

7. A method of treating a patient having acute respiratory distress syndrome (ARDS) comprising administering to the patient an effective amount of marinobufagenin (MBG) antagonist.

8. The method of claim 6, wherein the MBG antagonist is resibufagenin (RBG).

9. The method of claim 6, wherein the MBG antagonist is administered intravenously or by inhalation.
